(19) United States
(12) Patent Application Publication
(10) Pub. No.: US 2022/0233301 A1
(43) Pub. Date: Jul. 28, 2022

(54) Tendon stretching mechanism

(71) Applicant: Christopher Stevens, Oro Valley, AZ (US)

(72) Inventor: Christopher Stevens, Oro Valley, AZ (US)

(21) Appl. No.: 16/974,243

(22) Filed: Jan. 26, 2021

(51) Int. Cl. A61F 2/08

(52) U.S. Cl. CPC A61F 2/0805; A61F 2/0811

(57) ABSTRACT

A tendon stretching mechanism which engages the torn tendon using sutures. A second end of the sutures is exposed through the skin. Using a tensioning mechanism, a selectively pull is applied to the second end of the sutures to gradually stretch the tendon. This stretching of the tendon may take a week or more or until the tendon has been sufficiently elongated for reattachment to the bone. The tensioning mechanism is either manually operated or is automatic.

### BACKGROUND OF THE INVENTION

This invention relates generally to rotator cuff tear repairs and the like, and more particularly to extension and elongating of the torn tendon from such injury.

While the present invention discusses the repair of a rotator cuff tear, the invention is not limited to this injury but is intended to include all injuries in which the tendon has been torn from a bone and must be reattached.

A rotator cuff tear is an injury where one or more of the tendons of the rotator cuff of the shoulder are torn free from the bone. When this occurs, there should be pain (getting worse with movement) and weakness in the shoulder. Obviously, daily activities which require the use of the arm such as brushing of the hair or even putting on clothing can be difficult and painful.

Tears often occur as a result of a traumatic even can occur gradually over time. Contributing factors such as repetitive activities, smoking, and family history have been noted. Identifying the problem is usually done through physical examination and/or medical imaging.

While the rotator cuff is made up of the supraspinatus, infraspinatus, teres minor and subscapularis, is it usually the supraspinatus which is most commonly affected.

Conservative treatment of the injury ranges from pain medications (e.g. NSAIDs), physical therapy, injections, and activity modifications. In severe or recalcitrant cases, surgery is often the treatment of choice.

If the patient delays in getting the surgery, a complete tear of the tendon allows the tendon to retract and pull completely away from the bone. During surgery, the tendon must then be elongated by manual stretching. If the stretching length is too much, excessive force by the surgeon can cause further damage to the tendon and limit the ability of the tendon to heal.

It is clear that there is a need for an efficient and safe mechanism to elongate a tendon allowing it to be reattached to the bone.

### SUMMARY OF THE INVENTION

The invention provides a tendon stretching mechanism which engages the tendon using sutures with a second end of the sutures being exposed through the skin. Using a tensioning mechanism, a selective pull is applied to the end of the sutures to gradually extend the tendon. This extending of the tendon may take a week or more or until the tendon has been sufficiently elongated for reattachment to the bone. The tensioning mechanism is ideally manually operated although an automatic version is also contemplated.

The device of the invention is an external device that sits on the lateral aspect of the shoulder against the skin. The device provides constant tension to the rotator cuff via antimicrobial impregnated sutures that are passed through the rotator cuff and secured to the external device.

Tension on the sutures is optionally increased at whatever frequency is determined to be necessary, such as twice a day. By turning a lever, pushing a button, or other type of actions, the tension is maintained on the sutures and can be increased.

In one embodiment, there is also a gauge which measures how much tension is being placed on the rotator cuff sutures (and by extension the tendon) to assure that excessive pressure/tension is not being applied and to know when additional tensioning is required by the patient. An alternative embodiment has a tension limiting mechanism that “clicks” when the maximum tension has been obtained.

In this context, to prevent over tensioning of the tendon, a variety of mechanisms are obvious to those of ordinary skill in the art, including, but not limited to those described in: U.S. Pat. No. 10,646,300, entitled “Ultrasonic Surgical Instrument with Transducer Locking Feature” issued May 12, 2020, to Roberson et al.; U.S. Pat. No. 10,660,722, entitled “Ultrasonic Surgical Instrument with Integral Shaft Assembly Torque Wrench”, issued May 26, 2020, to Lesko et al.; U.S. Pat. No. 10,744,626, entitled “Slip Type Torque Wrench” issued Aug. 18, 2020, to Lin; and, U.S. Pat. No. 10,751,862, entitled “T Handle Torque Wrench with Slip Function” issued Aug. 25, 2020, to Edmisten et al.; all which are incorporated hereinto by reference.

Tensioning of the device is done via a locking mechanism through which the sutures pass and operate to secure the sutures similar to a cable tie.

Protecting the skin from pressure is accomplished through the use of gel pads placed between the skin and tensioning mechanism itself. Ideally the gel pads cover approximately one third of the skin. In the preferred embodiment the gel pads are rotated three times a day to limit the areas of pressure on one particular area of the skin.

The surgical procedure is designed as a two stage procedure with the first stage being an arthroscopic debridement, possible suprascapular nerve release, subacromial bursa debridement and placement of the external fixator and sutures. The second stage is performed later, typically after two weeks, once sufficient tension has been applied to the tendon to elongate it. During this stage procedures performed include: distal clavicle excision (if warranted), subacromial decompression, removal of the external fixator and arthroscopic rotator cuff repair.

The reattachment of the tendon to the bone is well known in the art and is described in detail in U.S. Pat. No. 10,765,423, entitled “Suture Sleeve Patch and Methods of Delivery within an Existing Arthroscopic Workflow” issued Sep. 8, 2020, to Coleman; U.S. Pat. No. 10,653,515, entitled “Tendon Repair Apparatus and Method” issued May 19, 2020, to Fuller et al; U.S. Pat. No. 10,595,983, entitled “Constructs and Methods for Repairing a Tendon with a Reduced Risk of Reinjury” issued Mar. 24, 2020, to Ferguson et al; and, U.S. Pat. No. 10,265,156, entitled “Tenon Repair Implant and Method of Implantation” issued Apr. 23, 2019, to Van Kampen; all of which are incorporated hereinto by reference.

The mechanism of the present invention is made of materials which are safe for human use. Those of ordinary skill in the art recognize a variety of materials in this context, including, but not limited to those described in, U.S. Pat. No. 10,610,271, entitled “Orthopedic Surgical Implants” issued Apr. 7, 2020 to Anissian, incorporated hereinto by reference.

In more detail, the invention provides for a tendon stretching mechanism which has: an engagement mechanism configured to be secured to a tendon (preferably being multiple sutures) which have one end through an incision. A tensioning mechanism grasps the exposed ends of the multiple sutures external to the body and provides a selectively pull. The amount of tension being applied is defined by a user/patient over an extended period of time.

Those of ordinary skill in the art readily recognize a variety of acceptable techniques for exposing the sutures through the skin including, but not limited to U.S. Pat. No. 10,420,541, entitled “Thoracic Access Port” issued on Sep. 24, 2019, to Wilkins et al., incorporated hereinto by reference.

A variety of tensioning mechanisms are well known to those in the art, including but not limited to, U.S. Pat. No. 10,624,708, entitled “Auto Cable Tensioning System” issued Apr. 21, 2020, to Hunter; and U.S. Pat. No. 10,765,418, entitled “Knotless Dynamic Suture Tensioning Device and Methods” issued Sep. 8, 2020, to Foerster; both of which are incorporated hereinto by reference.

Ideally, the sutures are secured to the width of the tendon. This provides for an even pull across the entirety of the tendon to maintain the tendon's end in optimal condition for reattachment.

Ideally, the tendon stretching mechanism includes a base unit supporting the tensioning mechanism. The multiple sutures extend through a hole in the base unit and are grasped by the tension mechanism.

To minimize abrasion to the skin, at least one cushion is interposed between the base unit and the skin of the patient.

As noted, the tension is set initially during surgery by the surgeon and the patient increases the tension at defined intervals determined by the surgeon between the first and second procedures. It is the patient who applies the pressure using a handle or a pressure button. The handle optionally removable from the base unit or can be sprung into an accessible position by the patient.

Those of ordinary skill in the art recognize a variety of handles and mechanisms which can be used in this context, including, but not limited to those described in U.S. Pat. No. 10,478,207, entitled “Surgical Grasper” issued Nov. 19, 2019, to Lathrop, incorporated hereinto by reference.

The ideal arrangement is an operator/patient activated turn key being applied by the tensioning mechanism against the multiple sutures.

For safety purposes, either a gauge indicating the tensioning pressure being applied, or the tensioning mechanism includes a limiting mechanism which only allows the desired pressure to be applied (typically one to five pounds). The tensioning limiter emits a “clicking” when the desired pressure is applied.

An example of a tension limiting mechanism is described in U.S. Pat. No. 9,795,391, entitled “Tissue Compression Device with Tension Limiting Strap Retainer” issued Oct. 24, 2017, to Saatchi et al.; and U.S. Pat. No. 10,736,743, entitled “Handle Assembly for Implant Delivery Apparatus Comprising a Force Limiter, a Displacement limiter and/or brake frame Assembly” issued Aug. 11, 2020, to Lim et al.; all of which are incorporated hereinto by reference.

In some embodiments, a variety of mechanisms are available which allow for differing tensioning pressure limiters.

The invention together with various embodiments thereof will be more fully explained by the accompanying drawings and the following description thereof.

### DRAWINGS IN BRIEF

FIG. 1 illustrates the attachment to the torn tendon and then passing through the skin.

FIG. 2 illustrates the placement of the sutures onto the torn tendon.

FIGS. 3A and 3B illustrate the placement of the tensioning mechanism onto the patient.

FIGS. 4A, 4B, and 4C, illustrate various embodiments for patient application of the tension onto the torn tendon.

FIGS. 5A and 5B illustrate two different embodiments highlighting the attachment of the sutures to the tensioning mechanism.

### DRAWINGS IN DETAIL

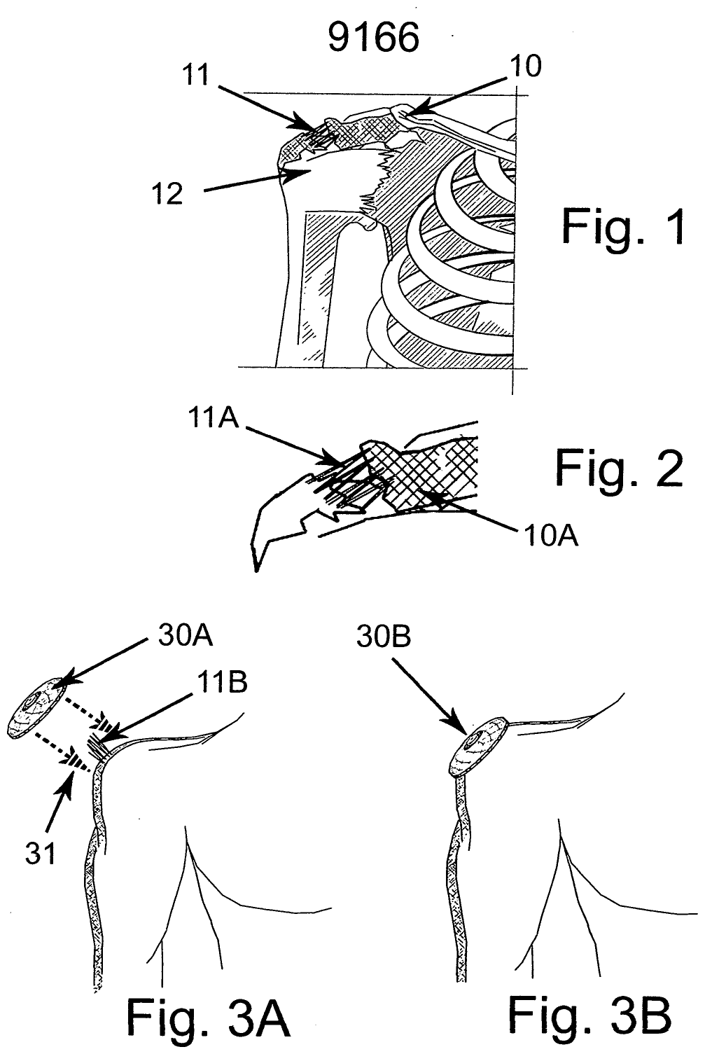

FIG. 1 illustrates the attachment to the torn tendon and then passing through the skin.

In this illustration, tendon 10 has been torn from bone 12 and has contracted sufficiently so that it cannot be readily reattached without possible further tearing of tendon 10. The first step is to secure the end of tendon 10 with multiple sutures 11.

FIG. 2 illustrates the placement of the sutures onto the torn tendon.

Sutures 11A are attached to the entire (or substantially the entire) width of the torn end of tendon 10A. This arrangement assures that when the apparatus of this invention is used to lengthen tendon 10A, the entire width is later available for reattachment to bone 12.

FIGS. 3A and 3B illustrate the placement of the tensioning mechanism onto the patient.

As shown in FIG. 3A, the ends of sutures 11B extend through the skin of the patient and are addressable by tensioning mechanism 30A as illustrated by arrows 31.

Once tensioning mechanism 30B is secure to the skin of the patient (with the sutures firmly attached thereto as will be explained later), proper tensioning of the sutures 11B may begin allowing the tendon to be gradually elongated.

Interposed between tensioning mechanism 30B and the skin of the patient, gel pads are provided to reduce pressure to the skin while the tension in the sutures is being gradually increased.

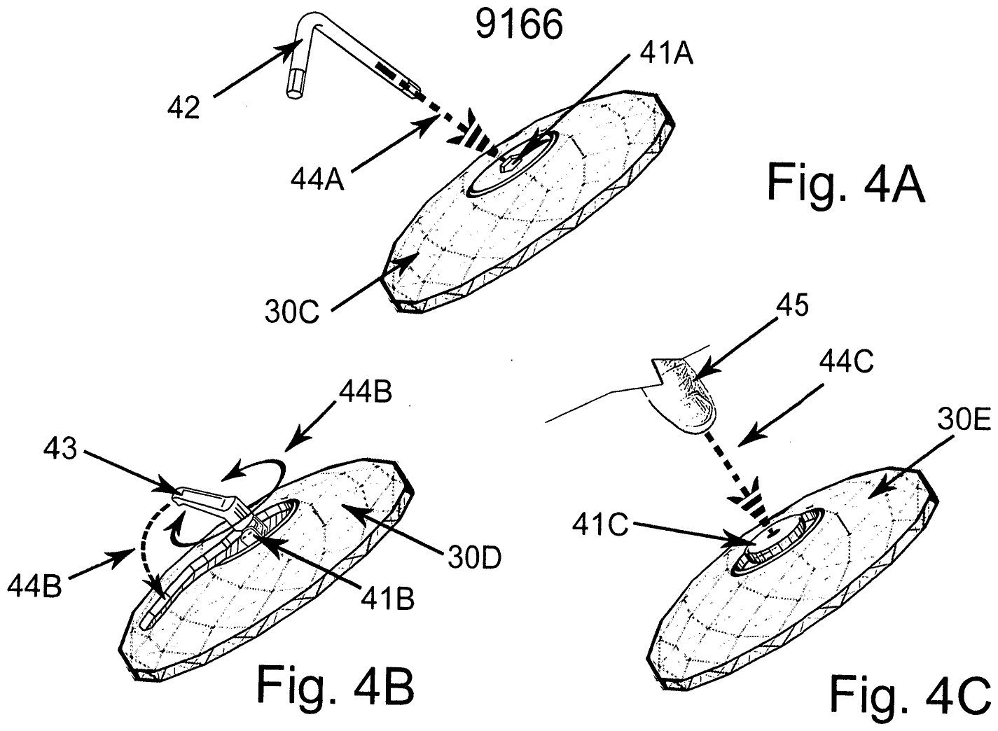

FIGS. 4A, 4B, and 4C, illustrate various embodiments for patient application of the tension onto the torn tendon.

Tensioning mechanism 30C, shown in FIG. 4A, has a center receptacle 41A which accepts handle 42 as indicated by arrow 44A. Handle 42 is used by patient to provide tensioning to the sutures, not shown.

The tensioning mechanisms are ideally smooth so that they do not “catch” or “snag” on clothing of the patient. The tensioning mechanism is to be worn for an extended period of time until the tendon has been stretched to the desired length, often three to five centimeters.

The embodiment of FIG. 4A allows the handle 42 to be removed and only used when tension is to be applied.

FIG. 4B uses an attached handle 43 which swivels, as indicated by arrows 44B upward to allow rotation as indicated by arrow 44B. This rotation of handle 43 also rotates the center mechanism 41B of the tensioning mechanism 30D. When not in use, handle 43 lays flat against the upper surface of tensioning mechanism 30D.

A handless embodiment is shown in FIG. 4C which allows patient 45 to press center button 41C of tensioning mechanism 30E. This pressure causes the tensioning of the tendon. In some embodiments, an electric motor or a wound spring is used to drive the tensioning mechanism in response to activation of the button 41C.

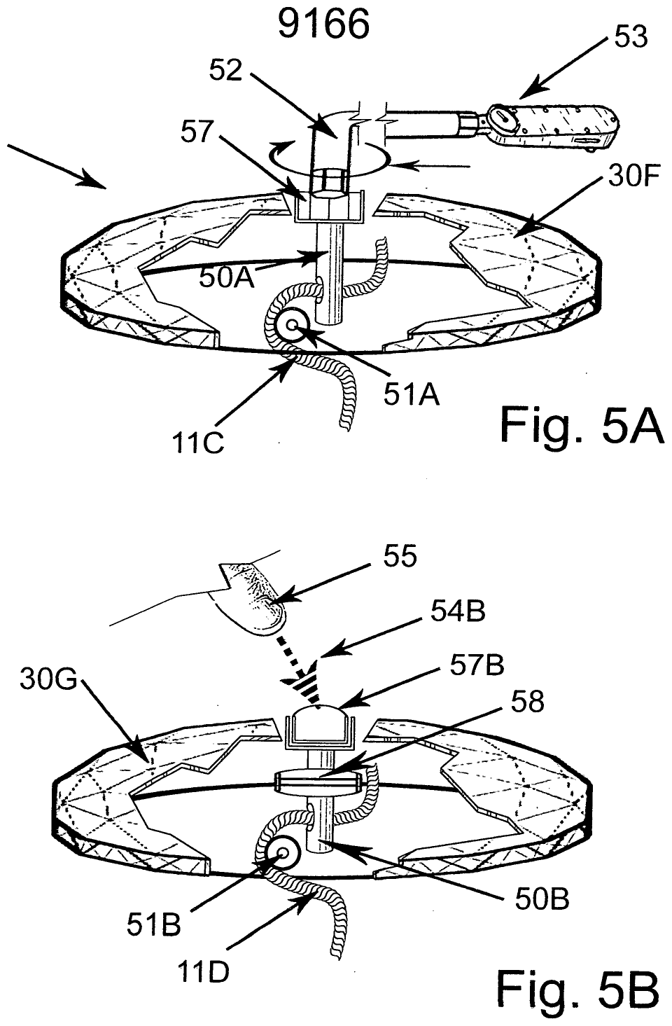

FIGS. 5A and 5B illustrate two different embodiments highlighting the attachment of the sutures to the tensioning mechanism.

Referencing FIG. 5A, in this embodiment, tensioning mechanism 30F is addressed by the patient using handle 52 which engages with a top receptacle 57 and is rotated as indicated by arrow 54A. Gauge 53, secured to the top of handle 52, provides feed back to the patient as to the amount of tension being applied. Gauge 53 helps to prevent over tensioning of the tendon.

Sutures 11C are fed into the tensioning mechanism around guide 51A to be inserted through pin 50A connected to top receptacle 57. As handle 52 is rotated, sutures 11C are wound around pin 50A to provide the desired tension on the tendon (not shown).

FIG. 5B uses a pressure 54B provide by the patient 55 which presses downward on button 57B. This pressure interacts with gear 58 to cause pin 50B to rotate, drawing sutures 11D onto pin 50B after the sutures 11D have passed by guide 51B.

It is clear that the present invention provides a safe and effective mechanism for the elongation of a tendon prior to reattachment.

What is claimed is:

1. A tendon stretching mechanism comprising:
a) an engagement mechanism configured to be secured to a tendon, said engagement mechanism having multiple sutures extending from the tendon and exiting through an incision; and,
b) a tensioning mechanism grasping the multiple sutures external to the body, said tensioning mechanism adapted to selectively pull, as defined by a user over an extended period of time, the multiple sutures.

2. The tendon stretching mechanism according to claim 1, wherein said engagement mechanism is secured to a width of the tendon.

3. The tendon stretching mechanism according to claim 2, further including a base unit supporting the tensioning mechanism, said base unit having a hole through which the multiple sutures extend to the tensioning mechanism.

4. The tendon stretching mechanism according to claim 3, wherein the base unit includes at least one cushion interposed between the base unit and skin of the patient.

5. The tendon stretching mechanism according to claim 4, wherein the tensioning mechanism includes an operator activated handle adapted to adjust tension being applied by the tensioning mechanism against the multiple sutures.

6. The tendon stretching mechanism according to claim 4, wherein the tensioning mechanism includes an operator activated push button adapted to adjust tension being applied by the tensioning mechanism against the multiple sutures.

7. The tendon stretching mechanism according to claim 6, wherein the tensioning mechanism includes a limiting mechanism adapted to maintain a tension of the multiple sutures below a selected level.

8. The tendon stretching mechanism according to claim 7, wherein the selected level is five pounds.

9. The tendon stretching mechanism according to claim 7, further including an audible indicator when the selected level has been reached.

10. A tendon stretching mechanism comprising:
a) multiple sutures secured to a tendon at a first end, a second end of the multiple sutures extending through an incision from a patient's body; and,
b) a patient operated tensioning mechanism secured to the second end of the multiple sutures, said patient operated tensioning mechanism selectively applying a pull on the second end of the multiple sutures.

11. The tendon stretching mechanism according to claim 10, wherein the first ends of the multiple sutures extend the width of the tendon.

12. The tendon stretching mechanism according to claim 11, further including:
a) a base unit supporting the tensioning mechanism, said base unit having a hole therein through which the second end of the multiple sutures extend to the tensioning mechanism; and,
b) a cushion interposed between the base unit and skin of the patient.

13. The tendon stretching mechanism according to claim 10,
a) wherein the patient operated tensioning mechanism includes an operator activated handle adapted to adjust tension along the multiple sutures; and being applied by the tensioning mechanism against the multiple sutures; and,
b) a limiting mechanism contained within the patient operated tensioning mechanism configured to maintain tension of the multiple sutures below a selected level.

14. The tendon stretching mechanism according to claim 13, wherein the selected level is five pounds.

15. The tendon stretching mechanism according to claim 13, further including an audible indicator when the selected level has been reached.

16. A tendon stretching mechanism comprising:
a) multiple sutures, a first end of the multiple sutures secured to a tendon, a second end of the multiple sutures extending through an incision from a patient's body;
b) a base unit positioned on the skin of the patient around the incision, said base unit having multiple cushions interposed between the base unit and the skin of the patient;
c) a tensioning mechanism mounted on the base unit and secured to the second end of the multiple sutures, said tensioning mechanism applying a selected pull on the multiple sutures.

17. The tendon stretching mechanism according to claim 16, wherein the first ends of the multiple sutures extend the width of the tendon.

18. The tendon stretching mechanism according to claim 17,
a) wherein the tensioning mechanism is patient operated and includes an operator activated button adapted to adjust tension along the multiple sutures; and,
b) a limiting mechanism contained within the patient operated tensioning mechanism configured to maintain tension of the multiple sutures below a selected level.

19. The tendon stretching mechanism according to claim 18, further including an audible indicator when the selected level has been reached.

20. The tendon stretching mechanism according to claim 17, wherein the tensioning mechanism automatically maintains a selected pull on the second end of the multiple sutures.
